(19) United States
(12) Patent Application Publication
(10) Pub. No.: US 2022/0049282 A1
(43) Pub. Date: Feb. 17, 2022

(54) Methods for Discovery of Antimicrobial Compounds

(71) Applicant: Northeastern University, Boston, MA (US)

(72) Inventor: Slava EPSTEIN, Dedham, MA (US)

(21) Appl. No.: 17/401,278

(22) Filed: Aug. 12, 2021

Related U.S. Application Data
(60) Provisional application No. 63/064,887, filed on Aug. 12, 2020.

(51) Int. Cl. C12Q 1/24; C12N 1/00; C12M 1/12

(52) U.S. Cl. CPC C12Q 1/24; C12M 25/14; C12N 1/00

(57) ABSTRACT

Methods are provided for inducing expression of cryptic genes in microbial cells. Microbial cells of a strain derived from a natural environment but grown in a laboratory culture, and having cryptic genes not expressed in the laboratory culture, are placed into a culture chamber having at least one nanoporous membrane. The culture chamber is then placed into a natural environment similar to the environment from which the strain was originally obtained. In the natural environment, the microbial cells are isolated in the culture chamber and exposed to inducers that diffuse into the culture chamber via the nanoporous membrane, and expression of the cryptic gene is induced.

### CROSS-REFERENCE TO RELATED APPLICATIONS

This application claims priority to U.S. Provisional Application No. 63/064,887, filed 12 Aug. 2020, which is incorporated by reference herein in its entirety.

### BACKGROUND

Microbes are a foundation of discovery of bioactive molecules because they produce an enormous variety of secondary metabolites and gene products, some with desirable activities rendering then useful as antibiotics, anti-inflammatory agents, anticancer drugs, and industrial enzymes. Systematic search for such microbial bioactive compounds started in the mid-20th century, with spectacular success. However, the advent of next generation sequencing technologies uncovered that microbes have genomic capacity far beyond that which had been discovered so far. The genomes of lab-culturable microbial strains contain a remarkable number of cryptic genes or gene clusters that apparently code for novel bioactive compounds or metabolic pathways for producing them. However, these compounds have never been discovered because such microbial strains either do not produce them under laboratory culture conditions at all, or do so at below detectable levels.

Over the last two decades, significant efforts have been made by both academia and industry to learn how to activate these cryptic genes. The approaches tried have included the following: 1) reshuffling the whole genome, e.g., by non-targeted mutagenesis; 2) heterologous cloning of the cryptic genes under known promoters; 3) screening synthetic libraries in hope to find natural inducers; and 4) mimicking the natural milieu by co-culturing the potential producer with another organism. Despite heavy investment, no single approved drug has resulted from this effort.

These cryptic genes and gene clusters are particularly interesting because they are expected to produce novel antibiotics and other compounds urgently needed to combat drug-resistant bacteria and other pathogens, as well as cancers. There is an urgent need for methods to induce expression of such cryptic microbial genes.

### SUMMARY

The present technology provides methods for controlled cultivation of microbes that possess non-expressed genes (cryptic genes) when grown in laboratory culture. When these microbes are placed into their natural environment under conditions that allow them to be exposed to chemical factors and physical conditions found in that environment, cryptic genes can become expressed and their gene products analyzed and/or isolated for use or for investigation. The technology thus reveals and makes available novel compounds and gene products with biological, medical, or industrial uses.

One aspect of the technology is a method of inducing expression of a gene in a microbial cell. The method includes the steps of: (a) providing (i) a laboratory-grown culture comprising the microbial cell, wherein the gene is not expressed, or is expressed at a low level, in the laboratory culture, and wherein the microbial cell descends from a microbial cell isolated from a natural environment; and (ii) a culture chamber configured for use in a natural environment, the chamber comprising at least one nanoporous membrane; (b) placing the culture in the chamber and sealing the chamber such that diffusion of microbial cells into or out of the chamber does not occur; and (c) placing the sealed chamber into a natural environment for a period of time whereby expression of said gene is induced.

The above described method can optionally include the following additional steps: (d) recovering the chamber from said natural environment; (e) removing one or more microbial cells from the recovered chamber; and (f) optionally culturing the one or more removed microbial cells in a laboratory environment. A further option is to include the step of: (g) analyzing expression of said gene or analyzing a product of said gene after step (e) or step (f). Such analyzing can include identifying or isolating a chemical compound resulting from said gene expression. Such analyzing can include screening a chemical compound resulting from said gene expression for a biological activity, such as inhibition of growth of another microbial cell or a cancer cell, or toxicity towards another microbial cell or a cancer cell.

The present technology can be further summarized by the following list of features.

1. A method of inducing expression of a gene in a microbial cell, the method comprising the steps of:

(a) providing (i) a laboratory-grown culture comprising the microbial cell, wherein the gene is not expressed, or is expressed at a low level, in the laboratory culture, and wherein the microbial cell descends from a microbial cell isolated from a natural environment; and (ii) a culture chamber configured for use in a natural environment, the chamber comprising at least one nanoporous membrane;

(b) placing the culture in the chamber and sealing the chamber such that diffusion of microbial cells into or out of the chamber does not occur; and

(c) placing the sealed chamber into a natural environment for a period of time whereby expression of said gene is induced.

2. The method of feature 1, further comprising:

(d) recovering the chamber from said natural environment;

(e) removing one or more microbial cells from the recovered chamber; and

(f) optionally culturing the one or more removed microbial cells in a laboratory environment.

3. The method of feature 2, further comprising:

(g) analyzing expression of said gene or analyzing a product of said gene after step (e) or step (f).

4. The method of feature 3, wherein said analyzing comprises identifying or isolating a chemical compound resulting from said gene expression.
5. The method of feature 3 or feature 4, wherein said analyzing comprises screening a chemical compound resulting from said gene expression for a biological activity, such as inhibition of growth of another microbial cell or a cancer cell, or toxicity towards another microbial cell or a cancer cell.
6. The method of any of the preceding features, wherein expression of a plurality of genes is induced.
7. The method of feature 6, wherein said plurality of genes encode enzymes that carry out a metabolic pathway or portion thereof in said microbial cell.
8. The method of any of the preceding features, wherein the microbial cell is selected from the group consisting of a bacterial cell, a fungal cell, an archaeal cell, and a protist cell.
9. The method of any of the preceding features, wherein a chemical compound is identified or isolated, and wherein said compound has an antimicrobial, anti-inflammatory, antiviral, or anticancer activity.
10. A gene, gene product, or chemical compound identified as a result of carrying out the method of any of the preceding features.
11. The gene product of feature 10, wherein the gene product is an enzyme useful in an industrial process.
12. The method of any of the preceding features, wherein step (c) further comprises altering the natural environment.
13. The method of feature 12, wherein said altering comprises removal of a nutrient from the natural environment, addition of a chemical agent to the natural environment, increasing and/or decreasing a temperature of the natural environment, adding an antibiotic to the natural environment, introduction of a microbial species to the natural environment, changing a pH of the natural environment, addition of a chelating agent to the natural environment, addition of a mutagen to the natural environment, irradiation of the natural environment with electromagnetic radiation, or a combination thereof.
14. The method of any of the preceding features, wherein the at least one nanoporous membrane comprises pores having a diameter less than about 200 nm.
15. The method of any of the preceding features, wherein the period of time in (c) is at least about 1 day, 1 week, or 1 month.
16. A kit for inducing expression of a gene in a microbial cell, the kit comprising:

a culture chamber configured for use in a natural environment, the chamber including a chamber for incubation of a microbial cell culture and one or more nanoporous membranes; and instructions for performing the method of any of the preceding features.

### BRIEF DESCRIPTION OF THE DRAWINGS

FIG. 1 is a schematic representation of a method for discovering chemical compounds produced by microbes incubated in a natural environment.

FIG. 2 shows a flow diagram of an exemplary method for discovering chemical compounds or biomolecules produced by microbes incubated in a diffusion chamber.

FIG. 3 shows at the left side a schematic representation of a cross-sectional view of a diffusion chamber device, and at the right side a photo of an actual diffusion chamber device.

### DETAILED DESCRIPTION

The present technology provides methods to induce silent or cryptic genes of laboratory grown microbial species that were previously obtained from a natural environment. The technology awakens genes that embody adaptations of a microbial species for life in a natural environment, yet are missing from the laboratory culture conditions, by exposure to naturally occurring environmental conditions. Such environmental conditions are believed to activate the expression of microbial genes and clusters of genes through the presence of nutrients and chemical inducer compounds, as well as chemical and physical conditions including temperature, pH, and the presence or absence of ionic species and environmental toxins. Other environmental conditions that can activate gene expression include the presence in the environment of other microbial species or varieties, which may act synergistically, antagonistically, or competitively with the microbial cells under investigation, and which may form an evolved community or network containing multiple microbial species, both friendly and antagonistic. The technology offers methodology for controlled cultivation of microbes with cryptic genes in their natural environment, under conditions found in nature that cause the cryptic genes to be expressed, either with or without further intervention by a practitioner of the method. Conditions found in the natural environment, such as competition with other microbial species, often are alone sufficient to induce the expression of cryptic genes, without the introduction of artificial conditions or alteration of the environment. The result is that novel and potentially valuable bioactive and other useful molecules are produced, discovered, and further utilized.

Many genes of microbes such as bacteria become inactive or unexpressed under laboratory conditions due to the artificial nature of such conditions, under which there is limited and artificial exposure to nutrients, chemical factors, physical conditions, and other microbes compared to a natural environment. Many genes uncovered by sequencing are valuable adaptations for existence of these microbes in nature, but are not useful in laboratory culture. As a result, such genes become inactive and unexpressed in laboratory culture, possibly resulting in functional loss of entire metabolic pathways and resulting biochemical products due to dormancy of the genes controlling such pathways or reduced or absent expression of enzymes or even structural proteins. The present technology is based on the insight that exposure to the chemical, physical, and/or biological factors missing from laboratory culture but present in a natural environment will suffice to allow many cryptic genes to become re-activated, which allows analysis, identification, isolation, production, and use of new biochemical products.

Methods of the present technology require placing microbial cells from a laboratory culture, and therefore expected or known to possess cryptic genes, into a growth chamber that is configured for placement into a natural environment and exposure of the cells to chemical factors from the environment, including small molecules and macromolecules from the environment, but not other cells from the environment. Suitable diffusion chambers and methods for growing microorganisms with unknown nutrient requirements in their natural setting have been described. See, e.g., U.S. Pat. No. 7,011,957 entitled “Isolation and Cultivation of Microorganisms from Natural Environments and Drug Discovery Based Thereon”; U.S. Pat. No. 9,249,382, entitled “Devices and Method for the Selective Isolation of Microorganisms”; and WO 2016/187622A1, entitled “Method and Device for Cultivation and Analysis of Novel Microbial Species with Unknown Growth Requirements”; the contents of each of which are hereby incorporated by reference in their entirety. Such growth chambers can be directly used or repurposed for practicing the present methods.

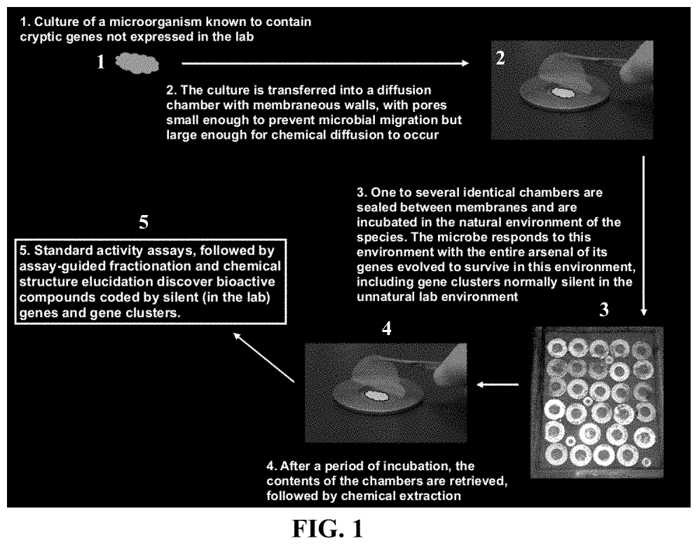

FIG. 1 presents a schematic diagram of an example of a method of the present technology. The method includes the following five steps.

In Step 1 a culture of microorganisms known or suspected to contain one or more cryptic genes of interest is provided. For example, the microorganisms can be laboratory-cultivated bacteria. As used herein, “laboratory-cultivated” microorganisms or microorganisms grown in a “laboratory environment” refers to microorganisms grown in controlled, artificial, non-naturally occurring conditions, such as culture conditions employing artificial or synthetic liquid or solid media containing one or more selected nutrients, which may be extracted from natural materials or synthesized, and a controlled pH. Generally, such conditions also involve incubation in an incubator or culture vessel at controlled temperature and/or atmospheric conditions, but culturing and growth of the cells do not need to be performed in a laboratory. Preferably the culture contains only a single species or strain of microbial cells, which are genetically identical or nearly genetically identical (i.e., the species or strain may contain genetic diversity typical for the population size). In certain embodiments, two or more different species or strains can be combined and placed together into the culture chamber. If desired, the species or strain of microbial cells used for the method can be confirmed as having low or no expression of targeted cryptic genes of interest. Gene expression can be measured by measuring an amount of a gene product, an expressed protein, or an amount of mRNA for a specific gene and comparing to expression of other genes or to expression of all genes for the cells grown under laboratory conditions.

In Step 2, cells from the culture are transferred to the inner space of diffusion chamber 40 having one or more porous membrane walls (for example, having two porous membrane walls). The pores of the membranes can have average diameters in the nanometer range, which allows diffusion of small molecules and macromolecules but not movement of microbial cells through the pores.

Step 3 requires incubating the chamber in a natural environment, which may be the natural environment from which the species or strain of microbial cells was originally found or isolated, or may be a different natural environment. As used herein a “natural environment” is a naturally occurring space and the conditions found therein, such as soil, a natural body of water, rock, sand, sediment, air, or a surface or interior of an animal (including a human) or a plant. A natural environment may be terrestrial or extraterrestrial. In certain embodiments, the natural environment can be a simulated natural environment including one or more extracts, isolates, or partially purified compositions (and in certain embodiments including individual purified or synthetic molecular components) obtained from a natural environment and added to a laboratory culture condition. Physical attributes of a natural environment can include, for example, vacuum, pressure, temperatures causing freezing or boiling of water, and any form of electromagnetic radiation or presence of high energy particles. Chemical attributes of a natural environment can include, for example, nutrients, growth factors, signaling compounds or nucleic acids from neighboring species. Cryptic genes of the microbial cells are expected to be activated during this incubation. The time of incubation can be selected according to need or as desired. The optimum incubation time can be found by trial and error, for example.

In Step 4 the chamber contents containing the microbial cells with activated cryptic genes are harvested or sampled. For example, the cells can be transferred to a laboratory culture, or not, and can optionally be washed and/or separated from any extracellular material. The entire culture or a portion thereof can be chemically or physically extracted so as to obtain molecular components of the cells or extracellular material for further processing, analysis, and/or investigation. The entire culture or a portion thereof can optionally be homogenized with or without cellular disruption, or can be extracted with a solvent or chemical solution.

Step 5 includes the analysis, identification, and/or purification or other characterization by any chemical and/or physical means. For example, the harvested and optionally processed material obtained from Step 4 can be subjected to any type of spectroscopy, mass spectrometry, chromatography, microscopy in attempt to identify molecular components of interest and related to the activation of one or more cryptic genes. Activity or functional assays can be carried out in the presence of varying amounts of harvested, isolated, or purified material, such as growth inhibition of other microbial cells or cancer cells or inhibition or stimulation of biomedical or physiological pathways or functions of interest. The results can be used to guide further fractionation, purification, and chemical structure elucidation for gene products or metabolites of interest. The process results in discovery of new bioactive compounds produced as a result of the activation of cryptic genes.

Exposure of the microbial cells to their natural chemical environment brings into the diffusion chamber many components of the strain's natural environment, such as nutrients, growth factors, signaling compounds from neighboring species. The expression of genes or gene clusters of the cells inside the diffusion chamber reverts to a state similar to that of the same type of cells if naturally found in the environment. Thus, the genes that are otherwise silent in the laboratory culture can be activated without foreknowledge of the biochemical requirements to do so. In the examples above, cryptic genes can be phenotypically silent DNA sequences that are not expressed during the laboratory culture due to the highly artificial nature of such conditions, from the microbial perspective.

Once previously inactive gene clusters are activated, novel compounds produced by the products of such genes (e.g., enzymes and regulatory proteins) can then be identified using standard bioassay-guided fractionation of the contents of the diffusion chamber. As an example, a given microbial species produces in the lab a certain number of antimicrobial compounds, but has a genomic potential to produce other such compounds as well. Once grown in nature, extracts of the given microbial species or strain can be screened against a panel of test species resistant to the known compounds. Any species or strain passing this assay is a producer of a novel antimicrobial activity coded by genes “silent” in the lab culture.

Alternatively, a given species or strain can be genetically modified to lose the ability to produce already known compounds. Its growth in diffusion chambers may then be screened against any test strain, as any detected antimicrobial activity must be the ones previously unknown in the strain. The detected activities can be scaled up for further investigation and utilization by using multiple chambers, or learning how to cultivate the producer in the lab without losing the discovered activities, or else by elucidating the chemical structure of the novel activity and chemically synthesizing it.

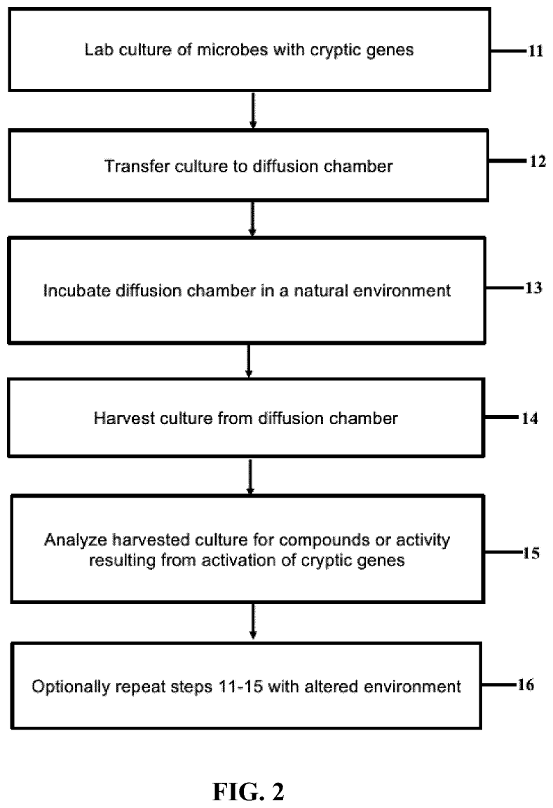

FIG. 2 illustrates a variation of the process of FIG. 1, activation of previously evolved cryptic (but phenotypically silent DNA sequences that are not expressed during the normal life cycle of an organism) that are a part of the genetic repertoire of the microorganism can optionally include use of a stress factor. The steps labeled 11-15 in FIG. 2 are equivalent to steps 1-5 of FIG. 1. However, additional step 16 of FIG. 2 allows for modification of the environment by an environmental modifier, which is then applied before, during, or after the incubation in an environment at step 13.

Optionally at least one environmental modifier is applied to the system during the incubation. Examples of modifiers are application and/or removal of an inducer, an antibiotic, a bioactive compound found in the environment, application of a test strain (i.e., a genetically different microorganism or a different strain of microorganism), moving the chamber from one environment to another, a change in pH of the environment, application of a chemical compound or biomolecule, introduction of another microbial species (i.e., that produces or secretes a chemical or biomolecule) near the system, application of electromagnetic radiation (e.g., UV/Vis light, gamma, X-ray), and a change in temperature. In many cases, diffusible small molecules from one strain can elicit production of secondary metabolites from a second strain. These diffusible molecules can, for example, act as nutrients, regulatory signals. or antibiotics. Examples of environmental modifiers also include withdrawal of a nutrient, adding an oxidative stress agent, adding one or more small molecules (chemical compounds), secondary metabolites or metabolites of other microbial cells, or adding or removing (or increasing or decreasing concentration of) naturally occurring chemicals of an environment such as a body of fresh or salt water, ground water, soil, rock, or even an extraterrestrial environment. Cryptic genes of the microbial cells are activated during this incubation.

Gene expression measurement can be achieved by quantifying levels of the gene product, which can often be a protein or a chemical compound. Two common techniques used for protein quantification include Western blotting and enzyme-linked immunosorbent assay or ELISA. Common techniques for chemical compound quantification are known in the art of analytical chemistry (e.g., quantitative NMR, chemiluminescent nitrogen detection).

Gene expression level can also be inferred by measuring the level of mRNA from a cryptic gene, which can be achieved using Northern blotting or serial analysis of gene expression (SAGE). Compared to other techniques for measuring gene expression, SAGE offers a significant advantage because it measures the expression of both known and unknown genes. Sometimes, when analyzing SAGE data, matches for certain tags cannot be found (e.g., by computer matching) in the sequence database. A lack of a (or any) match can indicate that the mRNA used to produce the unmatched tags is associated with a gene that has not been studied before. SAGE can be used to discover new genes transcribed.

Another technique for measuring mRNA is reverse transcription followed by quantitative polymerase chain reaction (RT-qPCR). Here, a DNA template is made from mRNA using reverse transcription. This template, which is called cDNA (complimentary DNA) is then amplified. As the DNA amplification proceeds, hybridization probes emit changing levels of fluorescence, which can be used to measure the original number of mRNA copies.

As used herein, a cryptic gene expressed at a low-level means a cryptic gene expression level calculated by the ratio between the expression of the cryptic gene (i.e., the gene of interest) and the expression of one or more reference genes (a known expressed gene). When measured as such a ratio, a low level means less than about 5% (less than about 5:100, cryptic gene:known gene), less than about 2% (less than about 2:100, cryptic gene:known gene), less than about 1% (less than about 1:100, cryptic gene:known gene), less than about 0.5% (less than about 0.5:100, cryptic gene:known gene), less than about 0.1% (less than about 0.1:100, cryptic gene:known gene), less than about 0.05% (less than about 0.05:100, cryptic gene:known gene), or less than about 0.01% (less than about 0.01:100, cryptic gene:known gene). The expression can be measured by comparing expression of a cryptic gene to expressions of all other combined genes (a weight comparison). When measured as such, a cryptic gene expressed at a low level means less than about 5% (weight/weight) of the expression of the cryptic gene/expressions of all other combined genes, less than about 2% (weight/weight) of the expression of the cryptic gene/expressions of all other combined genes, less than about 1% (weight/weight) of the expression of the cryptic gene/expressions of all other combined genes, less than about 0.5% (weight/weight) of the expression of the cryptic gene/expressions of all other combined genes, less than about 0.1% (weight/weight) of the expression of the cryptic gene/expressions of all other combined genes, less than about 0.05% (weight/weight) of the expression of the cryptic gene/expressions of all other combined genes, or less than about 0.01% (weight/weight) of the expression of the cryptic gene/expressions of all other combined genes.

The optional use of a stress factor can provide a controlled change or tunability to a natural environment. The presence of an active (noncryptic) gene may be disadvantageous in one environment, resulting in a strong selection for the cryptic state when the gene's product is not required. In another environment, due to a growth advantage, there might be a selection for the active state, for example, to provide the ability to utilize additional/different substrates. Depending on the environment, either the active or the cryptic state can be present under natural conditions. For example, an organism in which the genes for the metabolism of a specific compound are active will be handicapped in an environment in which toxic analogues of the compound are present. But conditions may exist where a nontoxic metabolite may be present as the sole nutritional source and it will be advantageous to activate the genes and be able to utilize that metabolite. Under such conditions, activation of a cryptic gene will be more advantageous compared to the induction of a normal but repressed gene in such cases. While the example method in FIG. 2 can provide for activation of previously evolved cryptic (but phenotypically silent DNA sequences that are not expressed during the normal life cycle of an organism), the technology contemplates use of an environmental modifier optionally including a mutagen, wherein the cryptic gene is activated by one or more mutational events, recombination, insertion elements, deletion, activation of different alleles that product different phenotype, or other genetic mechanisms.

The presently disclosed methods can induce expression of a cryptic gene in a microbial cell by placing and sealing the microbial cell in a culture chamber including at least one nanoporous membrane, placing the culture chamber into a natural environment that includes an inducer (or is suspected to include an inducer) for the cryptic gene, wherein the microbial cell is retained in the culture chamber (and microbial cells do not diffuse into the chamber), and the inducer diffuses into the culture chamber via the nanoporous membrane. Expression of said gene is induced. The technology contemplates the culture chamber can take a variety of shapes, configurations, and sizes, so long as the microbial cell is retained therein, and the inducer diffuses into the culture chamber. The presently disclosed methods may lead to a pipeline of novel chemical compounds with desirable activities that have been missed by previous technologies.

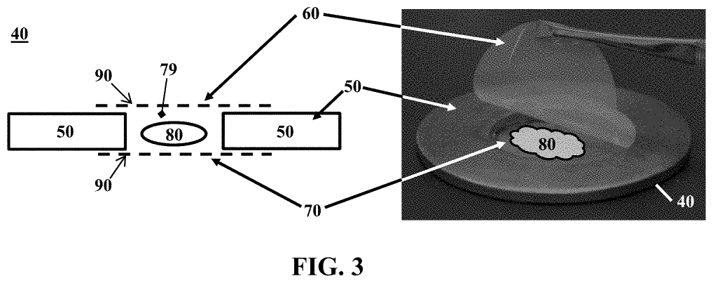

As discussed above, growth (or diffusion) chambers can be repurposed for practicing the present methods from, for example, U.S. Pat. Nos. 7,011,957, 9,249,382, and WO 2016/187622A1. Example photos of a diffusion chamber 40 are shown in FIG. 1 and in FIG. 3. A schematic representation of a cross-sectional view of the diffusion chamber device is shown at the left side of FIG. 3. Diffusion chamber 40 can be a sandwich structure with an inner space 79 enveloped by semi-permeable membranes 60, 70 on one or both sides. The membrane 70 is depicted with pores 90, but optionally, the membrane 70 can be without pores. One or both membranes can be configured to be transparent to a band of electromagnetic radiation, while shielding for other electromagnetic radiation. The substrate 50 can be any shape, and the thickness of the substrate can provide the inner space 79 (or substantially hollow space). The inner space is inoculated with a laboratory culturable microbial species 80 with known or unknown cryptic genes, i.e., genes that are not expressed under laboratory growth conditions, and the membranes separate the microbial cells from a natural or artificial environment. The membranes contain pores that are sufficiently small to prevent microbial cells from migrating in or out of the enclosed space—but still large enough to allow for diffusion of chemicals from the environment to occur. One or more sides of the chamber, or a portion thereof, can be made porous to chemical factors contained in the environment. The pores 90 can be less than 200 nm, less than 150 nm, or less than 100 nm in diameter) but large enough for diffusion of molecules to occur (e.g., greater than 1 nm, 2 nm, 3 nm, 4 nm, 5 nm, 6 nm, 7 nm, 8 nm, 9 nm, 10 nm, 12 nm, 15 nm, 20 nm, 25 nm, 30 nm, 40 nm, or 50 nm). The pores can be, for example, 0.02 microns (20 nm) in diameter. In another example, the pores can be smaller than the microbial cells in the culture chamber. Such a porous membrane is referred to herein as a “nanoporous” membrane. The chemicals in the environment of the growth chamber can be, for example, small molecules, metabolites of other microbial cells, naturally occurring chemicals of an environment such as a body of fresh or salt water, ground water, soil, rock, or even an extraterrestrial environment. The growth chamber containing the inoculated strain, located inside the chamber and covered by the membranes, is then returned to the environment, which can be, and preferably is, an environment from which the strain of microbial cells had originally been isolated.

As used herein, the term “about” refers to a range of within plus or minus 10%, 5%, 1%, or 0.5% of the stated value.

As used herein, “consisting essentially of” allows the inclusion of materials or steps that do not materially affect the basic and novel characteristics of the claim. Any recitation herein of the term “comprising”, particularly in a description of components of a composition or in a description of elements of a device, can be exchanged with the alternative expression “consisting of” or “consisting essentially of”.

What is claimed is:

1. A method of inducing expression of a gene in a microbial cell, the method comprising the steps of:
(a) providing (i) a laboratory-grown culture comprising the microbial cell, wherein the gene is not expressed, or is expressed at a low level, in the laboratory culture, and wherein the microbial cell descends from a microbial cell isolated from a natural environment; and (ii) a culture chamber configured for use in a natural environment, the chamber comprising at least one nanoporous membrane;
(b) placing the culture in the chamber and sealing the chamber such that diffusion of microbial cells into or out of the chamber does not occur; and
(c) placing the sealed chamber into a natural environment for a period of time whereby expression of said gene is induced.

2. The method of claim 1, further comprising:
(d) recovering the chamber from said natural environment;
(e) removing one or more microbial cells from the recovered chamber; and
(f) optionally culturing the one or more removed microbial cells in a laboratory environment.

3. The method of claim 2, further comprising:
(g) analyzing expression of said gene or analyzing a product of said gene after step (e) or step (f).

4. The method of claim 3, wherein said analyzing comprises identifying or isolating a chemical compound resulting from said gene expression.

5. The method of claim 3, wherein said analyzing comprises screening a chemical compound resulting from said gene expression for a biological activity, such as inhibition of growth of another microbial cell or a cancer cell, or toxicity towards another microbial cell or a cancer cell.

6. The method of claim 1, wherein expression of a plurality of genes is induced.

7. The method of claim 6, wherein said plurality of genes encode enzymes that carry out a metabolic pathway or portion thereof in said microbial cell.

8. The method of claim 1, wherein the microbial cell is selected from the group consisting of a bacterial cell, a fungal cell, an archaeal cell, and a protist cell.

9. The method of claim 1, wherein a chemical compound is identified or isolated, and wherein said compound has an antimicrobial, anti-inflammatory, antiviral, or anticancer activity.

10. A gene, gene product, or chemical compound identified as a result of carrying out the method of claim 1.

11. The gene product of claim 10 which is useful as an industrial enzyme.

12. The method of claim 1, wherein step (c) further comprises altering the natural environment.

13. The method of claim 12, wherein said altering comprises removal of a nutrient from the natural environment, addition of a chemical agent to the natural environment, increasing and/or decreasing a temperature of the natural environment, adding an antibiotic to the natural environment, introduction of a microbial species to the natural environment, changing a pH of the natural environment, addition of a chelating agent to the natural environment, addition of a mutagen to the natural environment, irradiation of the natural environment with electromagnetic radiation, or a combination thereof.

14. The method of claim 1, wherein the at least one nanoporous membrane comprises pores having a diameter less than about 200 nm.

15. The method of claim 1, wherein the period of time in (c) is at least about 1 day, 1 week, or 1 month.

16. A kit for inducing expression of a gene in a microbial cell, the kit comprising:
a culture chamber configured for use in a natural environment, the chamber including a chamber for incubation of a microbial cell culture and one or more nanoporous membranes; and
instructions for performing the method of claim 1.
